(19) United States
(12) Patent Application Publication
(10) Pub. No.: US 2019/0307720 A1
(43) Pub. Date: Oct. 10, 2019

(54) Pharmaceutical Compositions Containing Cannabis, Uses Thereof and Methods for Alleviating Stress and/or Anxiety

(71) Applicant: Exzell Pharma Inc., Markham (CA)

(72) Inventor: Pardeep Nijhawan, Markham (CA)

(21) Appl. No.: 16/375,926

(22) Filed: Apr. 5, 2019

(30) Foreign Application Priority Data
Apr. 6, 2018 (CA) 3000494

(51) Int. Cl. A61K 31/352; A61P 25/18; A61K 31/4415; A61K 31/51; A61K 31/525; A61K 31/714; A61K 36/87; A61K 36/258; A61K 31/455; A61P 25/22; A61K 31/05

(52) U.S. Cl. CPC A61K 31/352; A61P 25/18; A61K 31/4415; A61K 31/51; A61K 31/525; A61K 9/7023; A61K 36/87; A61K 36/258; A61K 31/455; A61P 25/22; A61K 31/05; A61K 31/714

(57) ABSTRACT

A pharmaceutical composition containing cannabis is provided for treating stress and/or anxiety in a patient. Also provided are a use of the pharmaceutical composition for treating stress and/or anxiety in a patient, and methods for treating stress and/or anxiety. The pharmaceutical composition preferably includes a combination of therapeutically effective amounts of one or more of the following medicinal ingredients: cannabis, an herb, a Vitamin B, and/or Vitamin C.

### FIELD OF THE INVENTION

The present invention relates to pharmaceutical compositions and methods for alleviating stress and/or anxiety.

### BACKGROUND OF THE INVENTION

According to the anxiety and depression association of America, there are approximately 40 million Americans over the age of 18 or 18.1% of the population who suffer from anxiety disorders. Although highly treatable, only 36.9% of those suffering receive treatment. The causes of anxiety disorders are complex and include genetics, brain chemistry, personality and life events.

Many individuals who suffer from this also suffer from bipolar disorders, eating disorders, headaches, irritable bowel syndrome (IBS), sleep disorders, substance abuse, chronic pain, stress and fibromyalgia.

As common as the disorder is, the number of treatment options is limited. The most accepted treatment is cognitive behaviour therapy (CBT). In combination with medication, CBT does help a significant number of patients, but many need to have regular sessions. The medications used are mostly antidepressants and do have significant side effects especially when used long term. The four classes of medication used are selective serotonin reuptake inhibitors (SSRIs), Serotonin-Norephinephrine Reuptake Inhibitors (SNRIs), Benzodiazepines, and Tricyclic Antidepressants (TCA). SSRIs are known to cause insomnia, sexual dysfunction, peptic ulcer disease and weight gain. SNRIs can cause stomach upset, insomnia, headaches, sexual dysfunction and increase in blood pressure. Benzodiazepines can cause drowsiness and can lead to dependency. TCAs also have significant side effects including orthostatic hypotension, constipation, urinary retention, dry mouth and blurry vision.

Other therapy options include meditation, yoga, exercise, acupuncture and transcranial magnetic stimulation. All of these have limitations in the successful treating patients.

Cannabis is known to help relieve stress and help with anxiety. However, the dosing is important in reaching this therapeutic effect. University of Illinois and University of Chicago researchers studied 42 patients and found that the group that had the low dose of tetrahydrocannabinol (THC) of 7.5 milligrams had lower stress and less negative feelings compared to the higher dose group of THCs (12.5 milligrams).

The initial feeling one has after using cannabis is that of relaxation. Once the drug is metabolized, patients typically experience paradoxical anxiety. Stress is therefore increased in patients after they have used cannabis.

What may be needed is a pharmaceutical composition of cannabis that treats stress and/or anxiety while minimizing occurrences and/or severity of paradoxical anxiety.

### SUMMARY OF THE INVENTION

According to an aspect of the present invention, there is disclosed a pharmaceutical composition for treating stress and/or anxiety in a patient. The pharmaceutical composition may preferably, but need not necessarily, comprise alone or in combination, therapeutically effective amounts of cannabis and/or herbs for use as a medicinal product prescribed by a physician, a health care practitioner or an over-the-counter product available at pharmacies, marijuana dispensaries and/or mass food stores.

According to an aspect of the invention, there is preferably disclosed a pharmaceutical composition for treating stress and/or anxiety in a patient. The pharmaceutical composition may preferably, but need not necessarily, additionally comprise therapeutically effective amounts of Vitamin B including, but not limited to, Vitamin B1 (Thiamine), Vitamin B2 (Riboflavin), Vitamin B3 (Niacinamide), Vitamin B5 (Pantothenic Acid), Vitamin B6 (Pyridoxine), Vitamin B9 (Folate), and/or Vitamin B12 (Cobalamin).

According to an aspect of the invention, there is preferably disclosed a pharmaceutical composition for treating stress and/or anxiety in a patient. The pharmaceutical composition may preferably, but need not necessarily, additionally comprise therapeutically effective amounts of Vitamin C (Ascorbic Acid).

According to an aspect of the invention, there is preferably disclosed a pharmaceutical composition for treating stress and/or anxiety in a patient. The pharmaceutical composition may preferably, but need not necessarily, additionally comprise therapeutically effective amounts of Melissa officinalis (lemon balm).

According to an aspect of the invention, there is preferably disclosed a pharmaceutical composition for treating stress and/or anxiety in a patient. The pharmaceutical composition may preferably, but need not necessarily, additionally comprise therapeutically effective amounts of Passiflora incarnate (Passion Flower) to have an anti-anxiety benefit.

According to an aspect of the invention, there is preferably disclosed a pharmaceutical composition for treating stress and/or anxiety in a patient. The pharmaceutical composition may preferably, but need not necessarily, additionally comprise therapeutically effective amounts of Vitis vinifera (grape).

According to an aspect of the invention, there is preferably disclosed a pharmaceutical composition for treating stress and/or anxiety in a patient. The pharmaceutical composition may preferably, but need not necessarily, additionally comprise therapeutically effective amounts of Withania somnifera (ashwaganda, Indian Ginseng).

According to an aspect of the invention, there is preferably disclosed a pharmaceutical composition for treating stress and/or anxiety in a patient. The pharmaceutical composition may preferably, but need not necessarily, additionally comprise therapeutically effective amounts of Punica granatum (pomegranate).

According to an aspect of the invention, there is preferably disclosed a pharmaceutical composition for treating stress and/or anxiety in a patient. The pharmaceutical composition may preferably, but need not necessarily, comprise therapeutically effective amounts of Cannabis in any of the following form cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, delta-9 tetrahydrocannabinol (THC or dronabinol), delta-8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or a combination thereof.

According to an aspect of the invention, there is preferably disclosed a pharmaceutical composition for treating stress and/or anxiety in a patient. The pharmaceutical composition may preferably, but need not necessarily, comprise a pharmaceutically acceptable excipient selected from any of the group consisting of: hydroxypropylcellulose, starch, silicon dioxide, gelatin, magnesium stearate, or microcrystalline cellulose.

According to an aspect of the invention, there is preferably disclosed a pharmaceutical composition for treating stress and/or anxiety in a patient. The pharmaceutical composition may preferably, but need not necessarily, comprise the form of a tablet, caplet, capsule, dermal patch, oil drops, powder or a suspension.

Other advantages, features and characteristics of the present invention, as well as methods of use and applications of the related elements of the pharmaceutical composition and formulation will become more apparent upon consideration of the following detailed description and the appended claims, the latter of which are briefly described hereinbelow.

### DETAILED DESCRIPTION OF THE PREFERRED EMBODIMENTS

The description that follows, and the embodiments described therein, is provided by way of illustration of an example, or examples, of particular embodiments of the principles and aspects of the present invention. These examples are provided for the purposes of explanation, and not of limitation, of those principles and of the invention.

It should also be appreciated that the present invention can be implemented in numerous ways, including as a use of the pharmaceutical composition or a method for treating stress and anxiety and/or symptoms associated with stress and anxiety. In this specification, these implementations, or any other form that the invention may take, may be referred to as uses or methods. In general, the order of the steps of the disclosed methods may be altered within the scope of the invention.

In this disclosure, a number of terms are used. The following definitions of such terms are provided.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

As used herein, the skilled reader may generally understand the term “stress” to generally mean a physical, mental or emotional factor that causes bodily or mental tension. Persons skilled in the relevant art will understand that stress can cause or influence the course of many medical conditions including psychological conditions such as depression and anxiety. Medical problems associated with stress, including chronic stress, may include poor healing, irritable bowel syndrome, high blood pressure, poorly controlled diabetes amongst others. Persons skilled in the relevant art may also understand the term “stress” to include the emotional, physical, cognitive and/or behavioral symptoms of stress. Emotional symptoms of stress may include: becoming easily agitated, frustrated and moody; feeling overwhelmed; experiencing difficulty relaxing and quieting the mind; low self-esteem; avoiding others. Physical symptoms of stress may include: low energy; headaches; upset stomach including diarrhea, constipation and nausea; cardiovascular effects; insomnia; frequent colds and infections; loss of sexual desire; nervousness and shaking; dry mouth; clenched jaw. Cognitive symptoms of stress may include: constant worrying; racing thoughts; forgetfulness and disorganization; inability to focus; poor judgment; and being pessimistic or negative. Behavioral symptoms of stress may include: changes in appetite; procrastinating and avoiding responsibilities; increased use of alcohol, drugs, cigarettes; and exhibiting more nervous tendencies.

As used herein, the skilled reader may generally understand the term “anxiety” to generally mean a feeling of apprehension and fear, characterized by physical symptoms such as palpitations, sweating and feelings of stress. Persons skilled in the relevant art may also understand the term “anxiety” to include additional symptoms, including: nervousness, restlessness, or being tense; feelings of danger, panic or dread; rapid heart rate; rapid breathing or hyperventilation; increased or heavy sweating; trembling or muscle twitching; weakness and lethargy; difficulty focusing or thinking clearly about a topic; insomnia; digestive or gastrointestinal problems; strong desire to avoid anxiety triggers; obsessions about certain ideas; performing certain behaviours repeatedly.

As used herein, a person skilled in the relevant art may generally understand the term “treatment” to generally refer to an approach for obtaining beneficial or desired results. Beneficial or desired results can include, but are not limited to, prevention or prophylaxis, alleviation or amelioration of one or more symptoms or conditions, diminishment of the extent of a disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a person skilled in the relevant art may generally understand the term “therapeutically effective amount” to be an amount sufficient to effect treatment when administered to a subject in need of treatment. In the case of the embodiments of the present invention, a therapeutically effective amount can include, but is not limited to, an amount that treats stress and/or anxiety.

It will be understood by a person skilled in the relevant art that the compositions of the present invention can be formulated into pharmaceutical compositions for administration in a manner customary for administration of such materials using standard pharmaceutical formulation chemistries and methodologies, all of which are readily available to a person skilled in the relevant art. It will also be understood by a person skilled in the relevant art that such pharmaceutical compositions may include one or more excipients, carriers, stabilizers or other pharmaceutically inactive compounds, such as, but not limited to, wetting or emulsifying agents, pH buffering substances, hydroxypropylcellulose, starch, silicon dioxide, gelatin, magnesium stearate, microcrystalline cellulose and the like. Pharmaceutically acceptable salts can also be included therein. A thorough discussion of pharmaceutically acceptable excipients, vehicles and auxiliary substances is available in Remington's Pharmaceutical Sciences (Mack Pub. Co. N.J. 1991). Such pharmaceutical compositions can be prepared as oral or transdermal preparations. The therapeutically effective doses may vary according to body weight and the timing and duration of administration will be determined by specific clinical research protocols.

It will be understood by a person skilled in the relevant art that the term “dose” refers to the measured quantity of an agent, preferably a therapeutic agent, to be taken at one time to have a desired therapeutic effect(s). Preferably, “dose” as used herein means a specified quantity of a pharmaceutical or therapeutic agent provided in one or more administration. It will be further understood that a “dosage unit” or “dosage form” as used herein means a form in which the active agent is provided. It will be understood that any known dosage form may be employed with the present invention. These may include, solid dosage forms, liquid dosage forms, gel dosage forms, etc. The term “effective dose” or “effective dosage” is defined as an amount sufficient to achieve or at least partially achieve the desired effect. The term “therapeutically effective dose” is defined as an amount sufficient to cure or at least partially arrest the disease and/or one or more associated symptoms in a patient already suffering from the disease.

It will be understood by a person skilled in the relevant art that the term “administering” means providing a therapeutically active agent or composition to a subject, and includes, but is not limited to, administering by a medical professional and self-administering.

It will be understood by a person skilled in the relevant art that a “pharmaceutical agent” or “therapeutic agent” as used herein means a substance that provides a therapeutic effect when administered to a subject. “Pharmaceutical composition” means a mixture of substances suitable for administering to an individual that includes one or more pharmaceutical or therapeutically effective agents. The terms “active pharmaceutical ingredient” shall be understood to refer to a substance in a pharmaceutical composition that provides a desired effect.

The treatment of stress and/or anxiety in accordance with the present invention and as hereinafter defined for the purposes of this invention is directed to the treatment of stress and/or anxiety as defined herein, including alleviating and/or reducing one or more symptoms associated with stress and/or anxiety. Stress and/or anxiety, including the symptoms associated therewith, may be caused or mediated at least in part by neurotransmitters, nervous system, cardiovascular system, and/or other biological systems. In a preferred embodiment of the present invention, an agent or agents which can treat stress and/or anxiety including the range of symptoms associated with stress and/or anxiety is recommended. Since no single active ingredient is presently capable of treating stress and/or anxiety including the range of symptoms associated therewith, a pharmaceutical composition such as is described in the present invention is recommended.

Preferred embodiments of the present invention treat stress and/or anxiety including the one or more symptoms associated with stress and/or anxiety.

Preferably, the pharmaceutical compositions of the present invention may be provided with different active ingredients, different strengths and/or different formulations. Preferably, the pharmaceutical composition of the present invention comprises a therapeutically effective amount of cannabis. A person skilled in the relevant art would understand the term “cannabis” to refer to a genus of flowering plants in the family Cannabaceae which produce a group of chemicals called cannabinoids that produce physiological effects when administered to a patient. Persons skilled in the art will also readily appreciate that a cannabinoid is one of a class of diverse chemical compounds that acts on cannabinoid receptors (e.g., cannabinoid receptor type 1, cannabinoid receptor type 2) in cells that alter neurotransmitter release in the brain. In accordance with one or more preferred embodiments of the invention, the pharmaceutical composition may comprise cannabis-derived cannabinoids selected from the group consisting of: cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, delta-9 tetrahydrocannabinol (THC or dronabinol), delta-8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. In accordance with the present invention, compositions comprising a therapeutically effective amount of cannabis may preferably serve as a treatment of stress and/or anxiety. In a preferred embodiment of the pharmaceutical composition, the amount of CBD (cannabidiol) is greater than THC for improved treatment of anxiety. In accordance with some embodiments of the present invention, cannabis may be present in the composition in a particulate form with at least 50% by wt of the particles ranging in size from about 50 micron to about 2000 micron and wherein the particles comprise a cannabinoid, cannabinoid derivative, a terpene or a mixture thereof in a range of about 1 mg to about 30 mg of cannabinoid (CBD or THC) and most preferably from about 2.5 mg to about 10 mg per dose.

The pharmaceutical composition of the present invention may also comprise a therapeutically effective amount of one or more herbs, including but not limited to: Lemon balm (Melissa officinalis); Passion flower (Passiflora incarnata); pomegranate (Punica granatum); grape (Vitis vinifera); ashawaganda, Indian Ginseng (Somnifera); and combinations thereof.

In a preferred embodiment, the pharmaceutical composition of the present invention comprises a therapeutically effective amount of Lemon balm (Melissa officinalis). Persons skilled in the relevant art would understand the term “Lemon balm” to be a perennial herbaceous plant in the mint family Lamiaceae. Lemon balm leaves may have been used in the prior art as traditional medicine—as a tea, externally applied, or as an essential oil—in the treatment of disorders of the gastrointestinal tract, nervous system, liver, and bile. It has been reported that Lemon balm may also have been used in the prior art for cognitive purposes (e.g., the improvement of cognition and reduction in stress and anxiety) and to calm the nerves and to relax the body and hence help for stress and anxiety (See: NIH.gov Fact sheet Melissa officinalis). In accordance with the present invention, compositions comprising a therapeutically effective amount of Lemon balm may preferably serve to enhance the treatment of stress and/or anxiety. In accordance with some embodiments of the present invention, Lemon balm may be present in the composition from about 5 mg to about 1600 mg per dose and most preferably from about 15 mg to about 30 mg per dose.

In a preferred embodiment, the pharmaceutical composition of the present invention comprises a therapeutically effective amount of Passion flower (Passiflora incarnata). Persons skilled in the relevant art would understand the term “Passion flower” to be a perennial and member of the passionflower genus Passiflora. Passion flower may have been used in the prior art as a traditional remedy for anxiety, insomnia, hypertension, or as an antitussive. It has been reported that passion flower may exhibit sedative and anti-anxiety activity in tests involving laboratory animals. Human studies of Passiflora, in certain combination products, may have also demonstrated anti-anxiety and sedative properties. In addition, it has been reported that passion flower may have effects that are similar to benzodiazepines (e.g., diazepam [Valium™]) and monoamine oxidase inhibitors or MAOIs (e.g., selegiline). Passion flower has been reported to provide a calming, sleep inducing, and muscle spasm relieving effects. (See: NIH.gov Fact sheet Passiflora) In accordance with the present invention, compositions comprising a therapeutically effective amount of Passion flower may preferably serve to enhance the treatment of stress and/or anxiety. In accordance with some embodiments of the present invention, Passion flower may be present in the composition from about 25 mg to about 3000 mg per dose and most preferably from about 50 mg to about 100 mg per dose.

In a preferred embodiment, the pharmaceutical composition of the present invention comprises a therapeutically effective amount of pomegranate (Punica granatum). Persons skilled in the relevant art would understand the term “pomegranate” to be a fruit-bearing deciduous shrub or small tree in the family Lythraceae. It has been reported that pomegranate may have anxiolytic and antidepressant effects. (See: Riaz A, Khan R A. Behavioral effects of Citrus Limon and Punica Granatum combinations in rats. Metab Brain Di.2017 February: 32(1); 123-131) In accordance with the present invention, compositions comprising a therapeutically effective amount of pomegranate may preferably serve to enhance the treatment of stress and/or anxiety. In accordance with some embodiments of the present invention, pomegranate may be present in the composition from about 2 mg to about 1600 mg per dose and most preferably from about 2.5 mg to about 10 mg per dose.

In a preferred embodiment, the pharmaceutical composition of the present invention comprises a therapeutically effective amount of grape (Vitis vinifera). Persons skilled in the relevant art would understand the term “grape” to be the common grape vine and a species of Vitis containing flavonoids and stilbenoids. Persons skilled in the art will understand that flavonoids are polyphenols that can be classified as flavones, flavonols, flavanones, favanonols, flavanols or catechins, and anthocyanins. It may have been reported that flavonoids possess anxiolytic activities. (See: M Aslam, N Sultana. Evaluation of anxiolytic-like activity of Vitis vinifera juice in mice. Avicenna J Phytomed. 2016: May-June: 6(3):344-350) Persons skilled in the art will understand that stilbenoids are hydroxylated derivatives of stilbene and may have potential biological activity. In accordance with the present invention, compositions comprising a therapeutically effective amount of grape may preferably serve to enhance the treatment of stress and/or anxiety. In accordance with some embodiments of the present invention, grape may be present in the composition from about 1 mg to about 900 mg per dose and most preferably from about 2.5 mg to about 10 mg per dose.

In a preferred embodiment, the pharmaceutical composition of the present invention comprises a therapeutically effective amount of ashwaganda (Indian Ginseng or Withania somnifera). Persons skilled in the relevant art would understand the term “ashwaganda” to be a plant in the Solanaceae or nightshade family. It may have been reported that ashwaganda has been used in traditional medicine due to its antioxidant qualities and effect as an anxiolytic. Potential mechanisms of action, however, are not well understood. (See: Mishra L C, Singh B B, Dagenais S. Altern Med Rev. 2000 August; 5(4):334-46) In accordance with the present invention, compositions comprising a therapeutically effective amount of ashwaganda may preferably serve to enhance the treatment of stress and/or anxiety. In accordance with some embodiments of the present invention, ashwaganda may be present in the composition from about 25 mg to about 2000 mg per dose and most preferably from about 100 mg to about 200 mg per dose.

The pharmaceutical composition of the present invention may also comprise a therapeutically effective amount of one or more vitamins, including but not limited to: vitamin B; vitamin C; and combinations thereof.

Preferably, the pharmaceutical composition of the present invention comprises a therapeutically effective amount of vitamin B. A person skilled in the relevant art would understand the term “vitamin B” to comprise a class of water-soluble vitamins that play important roles in cellular metabolism. It has been reported in the prior art that vitamin B may have benefits in the treatment of stress and/or anxiety. In accordance with one or more preferred embodiments of the invention, the pharmaceutical composition may comprise vitamin B selected from the group consisting of: Vitamin B1(Thiamine); Vitamin B2 (Riboflavin); Vitamin B3 (Niacinamide); Vitamin B5 (Pantothenic Acid); Vitamin B6 (Pyridoxine); Vitamin B9 (Folate); Vitamin B12 (Cobalamin); and combinations thereof.

In a preferred embodiment, the pharmaceutical composition of the present invention comprises a therapeutically effective amount of folate (folic acid). A person skilled in the relevant art would understand that the term “folate” may comprise many forms of the vitamin—namely folic acid and its congeners, including tetrahydrofolic acid, methyltetrahydrofolate, methenylterahydrofolate, folinic acid, and folacin. It has been reported that folate (folic acid) has been shown to prevent depressive-like behaviour and improve stress in a study published by Budni. (See: Budni J, Zomkowski A D, Engel D et al Folic acid prevents depressive-like behavior and hippocampal antioxidant imbalance induced by restraint stress in mice. Exp Neurol 2013 February; 240:112-21) In the prior art, folic acid treatment has restored the activity of antioxidant enzymes and reduced lipid peroxidation in the hippocampus. In addition, folic acid in combination with Vitamin B12 may help improve cognitive performance and prevent brain shrinkage in stressed adults, which may be beneficial during aging. In accordance with the present invention, compositions comprising a therapeutically effective amount of folate may preferably serve to enhance the treatment of stress and/or anxiety. In accordance with some embodiments of the present invention, folate may be present in the composition from about 150 mcg to about 500 mcg per dose and most preferably from about 250 mcg to about 350 mcg per dose.

In a preferred embodiment, the pharmaceutical composition of the present invention comprises a therapeutically effective amount of Vitamin B2 (Riboflavin). A person skilled in the relevant art would understand that Vitamin B2 functions as a coenzyme. It has been reported that deficiency of Vitamin B2 (Riboflavin) can lead to increased anxiety and stress. In accordance with the present invention, compositions comprising a therapeutically effective amount of Vitamin B2 may preferably serve to enhance the treatment of stress and/or anxiety. In accordance with some embodiments of the present invention, Vitamin B2 may be present in the composition from about 1 mg to about 50 mg per dose and most preferably from about 10 mg to about 30 mg per dose.

In a preferred embodiment, the pharmaceutical composition of the present invention comprises a therapeutically effective amount of Vitamin B5 (pantothenic acid). A person skilled in the relevant art would understand that Vitamin B5 is used in the synthesis of coenzyme A and plays an important role in the synthesis of multiple neurotransmitters and steroids. It has been reported that deficiency can lead to anxiety and stress. (See: Kennedy D. B Vitamins and the Brain: Mechanisms, Dose and Efficacy-A Review. Nutrients, 2016 February: 8(2):68) In accordance with the present invention, compositions comprising a therapeutically effective amount of Vitamin B5 may preferably serve to enhance the treatment of stress and/or anxiety. In accordance with some embodiments of the present invention, Vitamin B5 may be present in the composition from about 1 mg to about 50 mg per dose and most preferably from about 10 mg to about 30 mg per dose.

In a preferred embodiment, the pharmaceutical composition of the present invention comprises a therapeutically effective amount of Vitamin B6 (Pyridoxine). A person skilled in the relevant art would understand that the term “Vitamin B6” refers to a group of chemically similar compounds that can be interconverted in biological systems and that an active form (pyridoxa 5′-phosphate) may serve as a coenzyme in enzyme reactions in amino acid, glucose, and lipid metabolism. Vitamin B6 has also been reported to be a potent antioxidant molecule and is typically ingested through diet (e.g., cereals, poultry, beef and potatoes) or nutritional supplementation. Reports have shown Vitamin B6 to increase resistance to abiotic stress in plants and may be beneficial for patients suffering from depression and anxiety. (See: Herve Vanderschuren, Svetlana Boycheva. Strategies for vitamin B6 biofortification of plants: a dual role as a micronutrient and a stress protectant. Front plant Sci, 2013:4:143) In accordance with the present invention, compositions comprising a therapeutically effective amount of Vitamin B6 may preferably serve to enhance the treatment of stress and/or anxiety. In accordance with some embodiments of the present invention, Vitamin B6 may be present in the composition from about 1 mg to about 50 mg per dose and most preferably from about 10 mg to about 30 mg per dose.

In a preferred embodiment, the pharmaceutical composition of the present invention comprises a therapeutically effective amount of Vitamin B3 (niacinamide). A person skilled in the relevant art would understand that “Vitamin B3” is a precursor of coenzymes including NAD and NADP which are needed in many metabolic processes. Persons skilled in the relevant art will also appreciate that niacinamide (nicotinamide) has been used in the prior art for treating niacin deficiency (pellagra), acne and has been reported to have a muscle relaxing quality that mellows anxiety. Reports suggest that niacinamide may have the similar effects on anxiety when compared to benzodiazepines. (See: J Prousky. Niacinamide's Potent role in Alleviating Anxiety with its Benzodiazepine-like Properties. Orthomolecular Health 2008.) In accordance with the present invention, compositions comprising a therapeutically effective amount of Vitamin B3 may preferably serve to enhance the treatment of stress and/or anxiety. In accordance with some embodiments of the present invention, Vitamin B3 may be present in the composition from about 10 mg to about 100 mg per dose and most preferably from about 10 mg to about 30 mg per dose.

In a preferred embodiment, the pharmaceutical composition of the present invention comprises a therapeutically effective amount of Vitamin B1 (Thiamine). A person skilled in the relevant art would understand that “Vitamin B1” is a coenzyme in the catabolism of sugars and amino acids. Persons skilled in the relevant art will also appreciate that thiamine has been used in the prior art to treat and prevent thiamine deficiency and disorders resulting from such deficiency. It has also been reported that Vitamin B1 (Thiamine) may improve Generalized Anxiety in a study done by Luong. (See: Loung K, Nguyen L. The Impact of Thiamine Treatment on Generalized Anxiety Disorder. IJCM, Vol 2 No4, September 2011.) In accordance with the present invention, compositions comprising a therapeutically effective amount of Vitamin B1 may preferably serve to enhance the treatment of stress and/or anxiety. In accordance with some embodiments of the present invention, Vitamin B1 may be present in the composition from about 1 mg to about 50 mg per dose and most preferably from about 10 mg to about 30 mg per dose.

In a preferred embodiment, the pharmaceutical composition of the present invention comprises a therapeutically effective amount of Vitamin B12 (Cobalamin). A person skilled in the relevant art would understand that “Vitamin B12” is a coenzyme involved in the metabolism of cells of the human body, especially affecting DNA synthesis and regulation, but also fatty acid metabolism and amino acid metabolism. Persons of skill in the relevant art will also appreciate that Vitamin B12 is involved in the normal functioning of the brain and nervous system via the synthesis of myelin and the formation of red blood cells. Vitamin B12 has been used to treat Vitamin B12 deficiency, cyanide poisoning, and hereditary deficiency of transcobalamin II. In accordance with the present invention, compositions comprising a therapeutically effective amount of Vitamin B12 may preferably serve to enhance the treatment of stress and/or anxiety. In accordance with some embodiments of the present invention, Vitamin B12 may be present in the composition from about 1 mcg to about 300 mcg per dose and most preferably from about 200 mcg to about 300 mcg per dose.

In a preferred embodiment, the pharmaceutical composition of the present invention comprises a therapeutically effective amount of Vitamin C (ascorbic acid). A person skilled in the relevant art would understand that “Vitamin C” is involved in the repair of tissue, enzymatic production of certain neurotransmitters, functioning of several enzymes, and immune system function. It has also been reported that Vitamin C is an antioxidant that is involved in anxiety, stress, depression, fatigue and mood state in humans. Prior art has shown that oxidative stress triggers neuropsychological disorders and that supplementation with vitamin C may help alleviate stress and/or anxiety in adults. (See: De Oliveira I J, de Souza V V, Motta V, Da-Silva S L. Effects of Oral Vitamin C Supplementation on Anxiety in Students: A Double Blind, Randomized Placebo-Controlled Trial. Pak J Biol Sci 2015 January; 18(1):11-8) In accordance with the present invention, compositions comprising a therapeutically effective amount of Vitamin C may preferably serve to enhance the treatment of stress and/or anxiety. In accordance with some embodiments of the present invention, Vitamin C may be present in the composition from about 50 mg to about 3000 mg per dose and most preferably from about 200 mg to about 300 mg per dose.

In preferred embodiments of the present invention, the combination of cannabis with herbs and/or vitamins provides advantageous outcomes as the pharmaceutical composition retains the therapeutic benefits of cannabis on stress and/or anxiety while minimizing its potential adverse effects.

The pharmaceutical composition preferably, but not necessarily, can be administered in a single or multiple daily doses when experiencing stress and/or anxiety. In preferred embodiments, a dose is taken once daily or twice daily. In preferred embodiments, a dosing regimen is continued until the feeling of stress and/or anxiety has abated. In some embodiments, the composition of the present invention may be administered once daily to prevent stress and/or anxiety.

In a preferred embodiment, the pharmaceutical composition of the present invention is used as a nutritional supplement to help patients who suffer from stress and/or anxiety. The pharmaceutical composition of the present invention will preferably minimize stress and help control anxiety in mild to moderate cases. Preferably, the disorders that may be treated by embodiments of the present invention include, but are not limited to, generalized anxiety disorders (GAD), panic disorder, panic attacks, agoraphobia, social anxiety disorder, separation anxiety, posttraumatic stress disorder (PTSD), and everyday stress. The pharmaceutical composition of the present invention is preferably formulated in multiple strengths to treat the symptoms associated with the above disorders. The pharmaceutical composition of the present invention is preferably formulated to use the minimal therapeutically effective doses of the active ingredients to maximize absorption in the gastrointestinal track (e.g., maximize bioavailability) and minimize side effects. In accordance with a preferred embodiment of the present invention, the pharmaceutical composition allows for a reduction in the dose of cannabis required to achieve the same therapeutic effect compared to cannabis administered to a patient in a traditional dosage form (e.g., smoking or inhalation).

In accordance with a preferred embodiment of the present invention, the pharmaceutical composition is formulated to contain cannabis comprising a higher dose of CBD (cannabidol) than THC (delta-9-tetrahydrocannabinol). An advantage of such formulations is an enhanced anti-anxiety effect.

In accordance with a preferred embodiment of the present invention, the administration of lower doses of cannabis (compared to cannabis administered in a traditional dosage form, for example, smoking or inhalation) combined with the vitamins and/or herbs disclosed herein is advantageous as it provides a therapeutic effect on stress and/or anxiety and a reduction in cravings for further use of cannabis that chronic cannabis users may experience.

In accordance with a preferred embodiment of the present invention, the combination of cannabis, vitamins and/or herbs disclosed herein is advantageous as it may provide an unexpected synergistic therapeutic effect on stress and/or anxiety.

In accordance with a preferred embodiment of the present invention, the combination of cannabis, vitamins and/or herbs disclosed herein is advantageous as it may unexpectedly allow for the use of a lower dose of cannabis to achieve a similar therapeutic effect when compared to cannabis administered to a patient in a traditional dosage form (e.g., smoking or inhalation).

In accordance with a preferred embodiment of the present invention, the combination of cannabis, vitamins and/or herbs disclosed herein is advantageous as it may allow for the use of a lower dose of cannabis to reduce the severity and/or number of potential cannabis-related adverse effects.

The pharmaceutical composition of the present invention is a formulation comprising cannabis in addition to various herbs and vitamins, either used as a medicine prescribed by a physician, a health care practitioner, or an over-the-counter product available in pharmacies, marijuana dispensaries, and mass food stores and will preferably treat stress and/or anxiety in patients.

In accordance with a preferred embodiment of the present invention, the pharmaceutical composition comprising cannabis, herbs and vitamins may be administered once to twice daily during stressful and anxious events. The pharmaceutical composition of the present invention delivers high concentrations of several herbs and vitamins including Vitamin B (Vitamin B1 (Thiamine), Vitamin B2 (Riboflavin), Vitamin B3 (Niacinamide), Vitamin B5 (Pantothenic Acid), Vitamin B6 (Pyridoxine), Vitamin B9 (Folate), and Vitamin B12 (Cobalamin), Vitamin C (Ascorbic acid), Passiflora incarnata (passion flower), Melissa officinalis (lemon balm), Vitis vinifera (grape), Punica granatum (pomegranate), Withania somnifera (ashwaganda, Indian Ginseng) and therapeutically effective concentrations of cannabis in any form without causing increased side effects. The combination of the foregoing herbs, vitamins and cannabis in a single formulation for the treatment of stress and/or anxiety is unique and has not been previously described.

### Example 1

The following example sets out a preferred formulation of the pharmaceutical composition in accordance with the present invention for the treatment of stress and/or anxiety.

| Medicinal Ingredients | Quantity | | Extract |
|---|---|---|---|
| Folate | 300.0 | mcg | |
| Melissa officinalis | 20.0 | mg | 8:1 DHE: 160 mg |
| Niacinamide | 25.0 | mg | |
| Pantothenic acid | 25.0 | mg | |
| Passiflora incarnata | 75.0 | mg | 6:1 DHE: 450 mg |
| Punica granatum | 5.0 | mg | 3:1 DHE: 15 mg |
| Riboflavin | 25.0 | mg | |
| Thiamine | 25.0 | mg | |
| Vitamin B12 | 250.0 | mcg | |
| Vitamin B6 | 25.0 | mg | |
| Vitamin C | 250.0 | mg | |
| Vitis vinifera | 5.0 | mg | 120:1 DHE: 600 mg |
| Withania somnifera | 150.0 | mg | 5:1 DHE: 750 mg |
| Cannabis (THC) | 7.5 | mg | |

In preferred embodiments of the invention, the formulation is allergen free (e.g., egg products, wheat (gluten) and dairy (lactose)). In addition, all compounds in the formulation are preferably gluten free.

### Example 2

### Background

A test formulation comprising a combination of folate (300.0 mcg), Melissa officinalis (20.0 mg), niacinamide (25.0 mg), patothenic acid (25.0 mg), Passiflora incarnata (75.0 mg), Punica granatum (75.0 mg), Riboflavin (25.0 mg), Thiamine (25.0 mg), Thiamine (25.0 mg), Vitamin B12 (250.0 mcg), Vitamin B6 (25.0 mg), Vitamin C (250.0 mg), Vitis vinifera (5.0 mg), Withania somnifera (150.0 mg), and Cannabis (THC) (7.5 mg) was prepared in a capsule dosage form to study the effectiveness of the combination on the treatment of stress and/or anxiety.

Objective:

To assess whether the test formulation described above is effective in the treatment of stress and/or anxiety.

Methods:

A 34 year old male experiencing stress and anxiety for the majority of his adult life has become a chronic cannabis user. The subject was administered the capsule twice daily for four (4) weeks and then qualitatively assessed for stress and/or anxiety.

Results:

The subject experienced stress and anxiety that was easier to manage and his cravings for further use of cannabis were decreased. No obvious adverse effects of the test formulation were identified.

Conclusions:

The test formulation may be effective in the treatment of stress and/or anxiety. Further studies may be needed to qualitatively and/or quantitatively assess improvement in stress and/or anxiety and to determine the preferred medicinal ingredients (including the preferred quantity of each) to include in the composition as well as the potential adverse effects associated with same.

The above description is meant to be exemplary only, and one skilled in the art will recognize that changes may be made to the embodiments described without departing from the scope of the invention disclosed. Modifications which fall within the scope of the scope of the present invention will be apparent to those skilled in the art, in light of a review of this disclosure, and such modifications are intended to fall within the appended claims.

This concludes the description of presently preferred embodiments of the invention. The foregoing description has been presented for the purpose of illustration and is not intended to be exhaustive or to limit the invention to the precise form disclosed. Other modification, variations and alterations are possible in light of the above teaching and will be apparent to those skilled in the art, and may be used in the design and manufacture of other embodiments according to the present invention without departing from the spirit and scope of the invention. It is intended the scope of the invention be limited not by this description but only by the claims forming a part hereof.

What is claimed is:

1. A pharmaceutical composition for treating stress and/or anxiety in a patient comprising a therapeutically effective amount of cannabis, an herb, Vitamin B, Vitamin C and pharmaceutically acceptable excipients.

2. The pharmaceutical composition of claim 1, wherein the cannabis is selected from the group consisting of: cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, delta-9 tetrahydrocannabinol (THC or dronabinol), delta-8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof.

3. The pharmaceutical composition of claim 2, wherein the herb is selected from the group consisting of: Lemon balm (Melissa officinalis); Passion flower (Passiflora incarnata); pomegranate (Punica granatum); grape (Vitis vinifera); ashawaganda, Indian Ginseng (Somnifera); and combinations thereof.

4. The pharmaceutical composition of claim 3 comprising: Lemon balm (Melissa officinalis) from about 5 mg to about 1600 mg per dose; Passion flower (Passiflora incarnata) from about 25 mg to about 3000 mg per dose; pomegranate (Punica granatum) from about 2 mg to about 1600 mg per dose; grape (Vitis vinifera) from about 1 mg to about 900 mg per dose; and/or ashawaganda, Indian Ginseng (Somnifera) from about 25 mg to about 2000 mg per dose.

5. The pharmaceutical composition of claim 3 comprising: Lemon balm (Melissa officinalis) from about 15 mg to about 30 mg per dose; Passion flower (Passiflora incarnata) from about 50 mg to about 100 mg per dose; pomegranate (Punica granatum) from about 2.5 mg to about 10 mg per dose; grape (Vitis vinifera) from about 2.5 mg to about 10 mg per dose; and/or ashawaganda, Indian Ginseng (Somnifera) from about 100 mg to about 200 mg per dose.

6. The pharmaceutical composition of claim 5, wherein vitamin B is selected from the group consisting of: Vitamin B1 (Thiamine); Vitamin B2 (Riboflavin); Vitamin B3 (Niacinamide); Vitamin B5 (Pantothenic Acid); Vitamin B6 (Pyridoxine); Vitamin B9 (Folate); Vitamin B12 (Cobalamin); and combinations thereof.

7. The pharmaceutical composition of claim 6 comprising: Vitamin B1 (Thiamine) from about 1 mg to about 50 mg per dose; Vitamin B2 (Riboflavin) from about 1 mg to about 50 mg per dose; Vitamin B3 (Niacinamide) from about 10 mg to about 100 mg per dose; Vitamin B5 (Pantothenic Acid) from about 1 mg to about 50 mg per dose; Vitamin B6 (Pyridoxine) from about 1 mg to about 50 mg per dose; Vitamin B9 (Folate) from about 150 mcg to about 500 mcg per dose; and/or Vitamin B12 (Cobalamin) from about 1 mcg to about 300 mcg per dose.

8. The pharmaceutical composition of claim 6 comprising: Vitamin B1 (Thiamine) from about 10 mg to about 30 mg per dose; Vitamin B2 (Riboflavin) from about 10 mg to about 30 mg per dose; Vitamin B3 (Niacinamide) from about 10 mg to about 30 mg per dose; Vitamin B5 (Pantothenic Acid) from about 10 mg to about 30 mg per dose; Vitamin B6 (Pyridoxine) from about 10 mg to about 30 mg per dose; Vitamin B9 (Folate) from about 250 mcg to about 350 mcg per dose; and/or Vitamin B12 (Cobalamin) from about 200 mcg to about 300 mcg per dose.

9. The pharmaceutical composition of claim 8, wherein the vitamin C is from about 50 mg to about 3000 mg per dose.

10. The pharmaceutical composition of claim 8, wherein the vitamin C is from about 200 mg to about 300 mg per dose.

11. The pharmaceutical composition of claim 10, wherein the cannabis comprises from about 1 to about 30 mg of a cannabinoid per dose.

12. The pharmaceutical composition of claim 10, wherein the cannabis comprises from about 2.5 mg to about 10 mg of a cannabinoid per dose.

13. Use of a pharmaceutical composition comprising a therapeutically effective amount of cannabis, an herb, Vitamin B, Vitamin C and pharmaceutically acceptable excipients for the treatment of stress and/or anxiety in a patient.

14. A method for treating stress and/or anxiety in a patient comprising administering to the patient, in one or more doses, a pharmaceutical composition comprising a therapeutically effective amount of cannabis, an herb, Vitamin B, Vitamin C and pharmaceutically acceptable excipients.
